(19) United States
(12) Patent Application Publication
(10) Pub. No.: US 2024/0398587 A1
(43) Pub. Date: Dec. 5, 2024

(54) DEVICE AND METHOD FOR MEASURING THE ABSOLUTE JOINT ANGLE OF A PROSTHETIC DEVICE

(71) Applicant: College Park Industries, Inc., Warren, MI (US)

(72) Inventors: Jacob Michael Drews, Washington, MI (US); Aaron John Taszreak, China, MI (US); Anna Mae Marlatt, Waterford, MI (US)

(21) Appl. No.: 18/679,233

(22) Filed: May 30, 2024

Related U.S. Application Data
(60) Provisional application No. 63/505,820, filed on Jun. 2, 2023.

(51) Int. Cl. A61F 2/64; A61F 2/50; A61F 2/68; A61F 2/76

(52) U.S. Cl. CPC A61F 2/64; A61F 2/76; A61F 2002/5006; A61F 2002/5038; A61F 2002/6863; A61F 2002/7625

(57) ABSTRACT

A prosthetic knee uses a hydraulic damper to regulate the rotation of the prosthetic knee joint. A magnetic rotary on-axis position sensor located at the joint between the upper and lower portions of the prosthetic knee measures the motion of the upper portion of the knee by detecting the magnetic field which is generated by a diametrically polarized magnet. A microprocessor correlates the measured motion to a knee joint angle, and adjusts the resistance provided by the hydraulic damper according to a corresponding stage of gait.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The present disclosure relates generally to sensor system for a hydraulic damper to regulate the rotation of a prosthetic joint, more specifically, a prosthetic knee joint.

### Description of the Related Art

Prosthetics are used to replace and restore the functionality of amputated natural body parts. Microprocessor-controlled prosthetic knees may use gearmotors for controlling the rotation of hydraulic valves. Present microprocessor-controlled prosthetic knees use a variety of methods for measuring the knee joint angle.

Some existing products measure the angle of the knee joint in order to determine modifications to the knee joint resistance depending on phase of gait and activity. In these devices, the upper bone, or thigh segment, pivots about an axial pin which connects the upper bone segment to the knee frame, lower bone, or shin segment. The shaft of the axial pin is a horizontal track which remains stationary during knee flexion and extension. A small magnet sits in said track. A hall effect sensor may be positioned on a circuit board at the front of the upper bone segment which measures the displacement of the magnet along the horizontal track. This displacement is correlated to an angle of the knee joint. This method is not ideal for determining the knee joint angle as it doesn't measure the angle directly at the source of rotation and it requires additional componentry. Furthermore, it requires additional moving parts, and the magnet can become loose from the track. Further, if debris ingresses the system, it will impact the magnet as it travels along the track. If the magnet can no longer readily slide along the track, the knee joint angle will no longer be detectable. When the knee angle is no longer detectable, the knee will no longer function or trigger properly.

Other existing devices use an induction sensor for determining the knee joint angle. Instead of measuring the displacement of a magnet along a track, the upper bone portion of these prosthetic knees are made of a metal material of variable thickness surrounding the axial pin. An induction sensor is positioned in front of the upper bone segment, and, as the upper bone rotates about the axial pin, the thickness of the material between the upper bone and axial pin varies and the induction sensor can detect the amount of material in proximity. This thickness of the metal material is correlated to the knee joint angle. While this method has the added advantage of measuring the knee joint angle directly from the pivot point and can lessen the power requirements needed, it is not ideal because that induction sensors are generally less accurate than position or displacement sensors.

In other known devices, a diametrically polarized magnet is located on the axial pin and a linear Hall Effect sensor is positioned in front of the upper bone segment. As the knee joint moves, the Hall Effect sensor detects the magnetic field presence. This magnetic field is correlated to a knee angle which is used for making decisions on the joint resistances. However, the use of linear Hall effect sensors in such devices are not ideal as they are limited to measuring only linear motion.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices, and methods a prosthetic device, for example a microprocessor-controlled prosthetic knee.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

One aspect of the present disclosure is directed towards a prosthetic joint, comprising a frame, a connector pivotally coupled to a proximal portion of the frame which is configured to pivot in an anterior-posterior direction of the frame about a pivot axis that extends in a medial-lateral direction of the frame. The frame and connector may both be portions of the joint. The prosthetic joint may further include a shaft extending along the pivot axis and extending through the connector and coupled to the frame on opposite sides of the connector. The prosthetic joint may further include a sensor attached to a side of the frame at or proximate the pivot axis. The prosthetic joint may further include a diametrically polarized magnet housed in a magnet cup located in a hollow portion of the shaft so that the magnet is centered along the pivot axis and the magnetic cup separates the magnet from the shaft. Rotation of the connector relative to the frame may simultaneously rotate the shaft, the magnet cup and the diametrically polarized magnet, and the sensor may be configured to measure a motion of the connector relative to the shaft by detecting a magnetic field generated by the magnet.

In some embodiments, the prosthetic joint is a prosthetic knee. In some embodiments, the prosthetic knee includes a hydraulic damper including a cylinder and piston configured for slidable travel within the cylinder.

In some embodiments, a distal portion of the frame of the prosthetic joint comprises a connector. In some embodiments, the connector may be a pyramid connector. In some embodiments, the pyramid connector may be configured to couple to a prosthetic device. In some embodiments, the prosthetic device may be a socket.

In some embodiments, the sensor of the prosthetic joint may be attached to a circuit board. In some embodiments, the sensor may be encased in a coating and/or an overmold configured to electrically isolate the circuit board. In some embodiments, the sensor may be encased within an enclosure, an overmold, and/or a coating configured to inhibit exposure of the sensor to environmental conditions. In some embodiments, the overmold may be comprised of resin.

In some embodiments, the magnet cup is configured to separate the diametrically polarized magnet from the shaft to not inhibit a magnetic field output of the diametrically polarized magnet.

In some embodiments, the prosthetic joint may further include a controller with a microprocessor configured to correlate the measured motion to a knee angle.

Another aspect of the present disclosure is directed towards a prosthetic knee including a hydraulic damper comprised of a cylinder and piston configured for slidable travel within the cylinder, a frame, a connector pivotally coupled to a proximal portion of the frame configured to pivot in an anterior-posterior direction of the frame about a pivot axis that extends in a medial-lateral direction of the frame, the frame and connector being portions of a joint. The prosthetic joint may further include a shaft extending along the pivot axis, through the connector and coupled to the frame on opposite sides of the connector. The prosthetic joint may further include a sensor attached to a side of the frame at or proximate the pivot axis, a diametrically polarized magnet housed in a magnet cup located in a hollow portion of the shaft so that the magnet is centered along the pivot axis, and the magnet cup separates the magnet from the shaft. Rotation of the connector relative to the frame may simultaneously rotate the shaft, the magnet cup and the diametrically polarized magnet, and the sensor may be configured to measure a motion of the connector relative to the shaft by detecting a magnetic field generated by the magnet.

In some embodiments, a distal portion of the frame of the prosthetic knee comprises a connector. In some embodiments, the connector may be a pyramid connector. In some embodiments, the pyramid connector may be configured to couple to a prosthetic device. In some embodiments, the prosthetic device may be a socket.

In some embodiments, the sensor of the prosthetic knee may be attached to a circuit board. In some embodiments, the sensor of the prosthetic knee is encased in a coating and/or an overmold configured to electrically isolate the circuit board. In some embodiments, the sensor of the prosthetic knee may be encased within an enclosure, an overmold, and/or a coating configured to inhibit exposure of the sensor to environmental conditions. In some embodiments, the overmold may be comprised of resin.

In some embodiments, the magnet cup of the prosthetic knee separates the diametrically polarized magnet from the shaft so as to not inhibit a magnetic field output of the diametrically polarized magnet.

In some embodiments, the prosthetic knee may further include a controller with a microprocessor configured to correlate the measured motion to a knee angle. In some embodiments, the controller may be configured to adjust a hydraulic resistance of the hydraulic damper based on the knee angle corresponding to the motion measured by the sensor.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

The following drawings are for illustrative purposes only and show non-limiting embodiments. Features from different figures may be combined in several embodiments.

FIG. 1 shows an example side view of a prosthetic knee.

FIG. 2 is an example side view of the axial pin shaft.

FIG. 3A shows an example of a knee angle circuit board.

FIG. 3B shows a close-up view of the knee angle circuit board of FIG. 3A.

FIG. 4 shows a front view of an example hydraulic damper of the prosthetic knee.

### DETAILED DESCRIPTION

The following detailed description is directed to certain specific embodiments of prosthetic devices and methods. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. The embodiments, examples of which are illustrated in the accompanying drawings, are set forth in detail below. Wherever possible, the same reference numbers are used throughout the drawings to refer to the same or like parts.

Microprocessor-controlled hydraulic prosthetic knees are an ideal solution for controlling the rotation of the prosthetic knee joint for above knee amputees. These systems employ one or more microprocessors and various sensors that detect, respond, and react to the bending of the knee joint by modifying the hydraulic resistance in both flexion and extension directions.

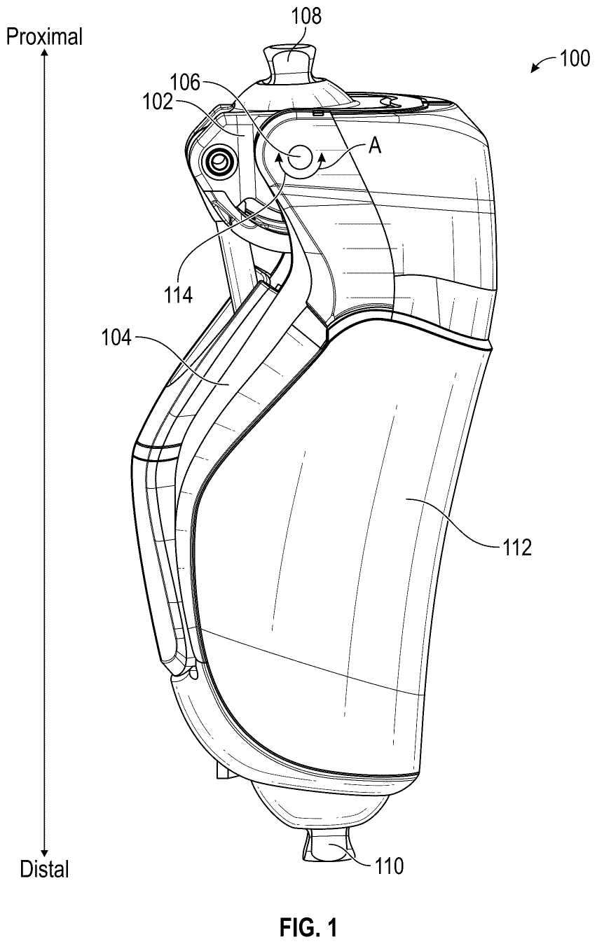

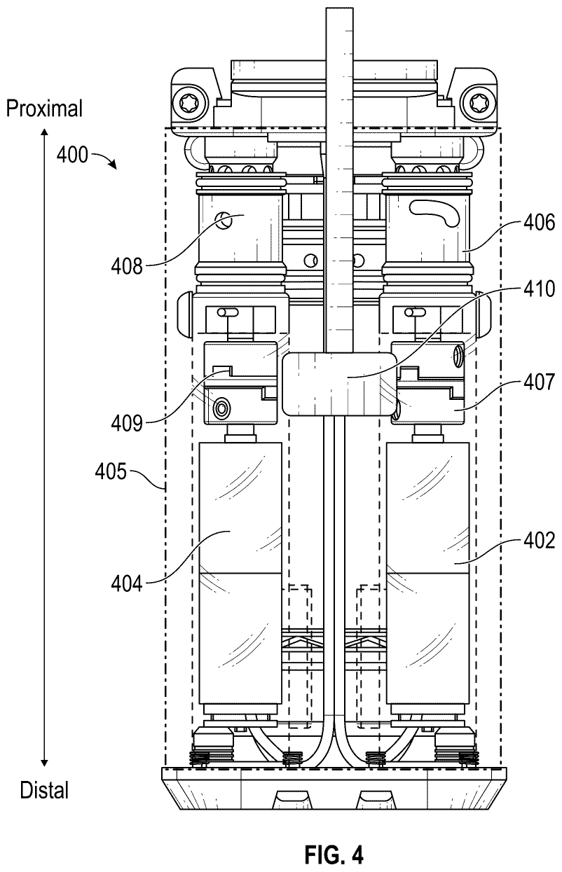

FIG. 1 shows an example side view of a prosthetic knee 100. The proximal end of the prosthetic knee 100 includes an upper frame portion 102, and the distal end of the prosthetic knee 100 includes and a lower frame portion 104. The upper frame portion 102 may include a proximal connector 108 (e.g., pyramid connector) at a proximal end for connecting the prosthetic knee 100 to an upper leg prosthetic device (e.g., a socket worn by an amputee over their upper leg). The lower frame portion 104 may include a distal connector 110 (e.g., pyramid connector) at a distal end for connecting the prosthetic knee to a lower leg prosthetic device (e.g., a pylon that couples to a prosthetic foot, a prosthetic foot, etc.). The prosthetic knee 100 may further include a hydraulic damper 400 housed within the body 112 of the lower frame portion 104, as described in more detail with respect to FIG. 4. The upper frame portion 102 and lower frame portion 104 are pivotably connected by a joint portion 114 to create a joint allowing for lateral and medial rotation about a pivot point 106 (i.e., in the direction shown by arrow A). An axial pin shaft 202 extends along the lateral axis of pivot point 106, through the joint portion 114 and is coupled to opposite sides (i.e., medial and lateral) of the lower frame portion 104. A joint portion 114 connects the upper frame portion 102 and the lower frame portion 104 at the pivot point 106. The bending of the hinge is regulated by the hydraulic damper 400. To determine when to make changes to the bending resistance of the prosthetic knee 100, a knee angle sensor (not shown) may be used to determine the state of the prosthetic knee 100 during gait and the bending resistance provided by the hydraulic damper 400 can be adjusted accordingly. In some embodiments, additional sensors may be required in order to confirm changes that should be made to the bending resistance of the prosthetic knee 100.

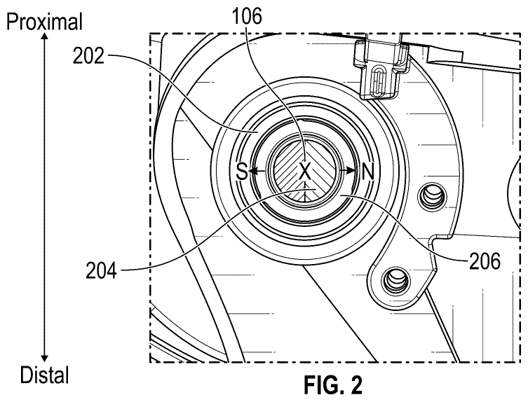

FIG. 2 is an example side view of the axial pin shaft 202. The axial pin shaft 202 may be made of metal (e.g., steel). A diametrically polarized magnet 204 is housed within an axial pin magnet cup 206 inserted into a hollow portion of the axial pin shaft 202 such that the magnet 204 is centered over the pivot point 106. As the upper frame portion 102 of the prosthetic knee 100 rotates, the axial pin shaft 202 and magnet 204 inside of the axial pin magnet cup 206 will also rotate (e.g., simultaneously). The axial pin magnet cup 206 advantageously separates the magnet 204 from the axial pin shaft 202 so as to not inhibit the magnetic field output of the magnet 204. The axial pin magnet cup may be made of any non-magnetic metal or plastic such as, for example, aluminum or nylon.

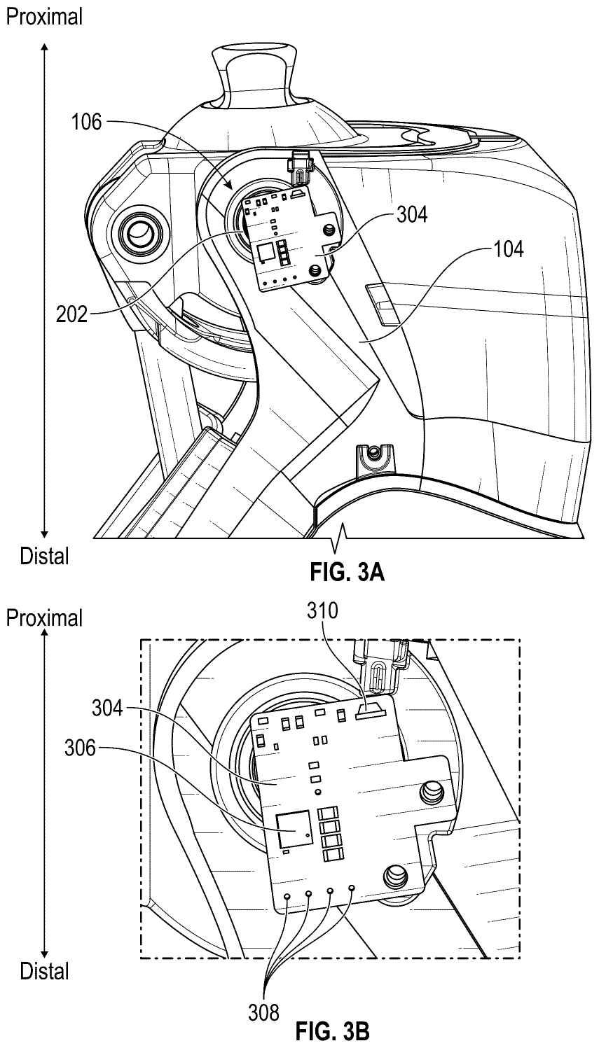

FIG. 3A shows an example of a knee angle circuit board 304 which is disposed between the axial pin shaft 202 and the joint portion 114. FIG. 3B is an example close up view of the knee angle circuit board 304. The knee angle circuit board 304 may include one or more of a knee angle sensor (not shown), one or more IC expanders 306, and one or more (e.g., one, two, three, four, etc.) solder connections 308. Solder connections 308 may be used to connect the knee angle circuit board 304 to a power source (e.g., an internal battery located in the prosthetic knee 100), ground, and a main circuit board of the prosthetic knee 100. The main circuit board may be located at another location of the prosthetic knee 100 (e.g., on a ventral side of the body 112) and may include, for example, one or more various sensors, a microprocessor, a Bluetooth module, and/or an inertial measurement unit (IMU).

The knee angle sensor (not shown) may be disposed on a back side of the knee angle circuit board 304 that faces the axial pin shaft 202. The knee angle sensor may be magnetic rotary position Hall Effect sensor, or any magnetic sensor capable of detecting magnetic field rotation. The knee angle sensor may be positioned directly in line with the pivot point 106 above the magnet 204 and be capable of measuring the motion of the upper frame portion 102 of prosthetic knee 100 by detecting the magnetic field generated by the magnet 204 during gait (e.g., during rotation of the knee joint). This motion can be correlated to a knee joint angle. The knee joint angle data is transferred from the knee angle sensor to the microprocessor of the main circuit board of the prosthetic knee 100 via an electric cable using the solder connections 308.

The microprocessor is capable of recognizing gait patterns from the information received from the knee angle sensor and various other sensors that may being included in the main circuit board of the prosthetic knee 100. The microprocessor reacts at transition points in the gait cycle by activating a gearmotor 402, 404 which adjusts a valve assembly in the hydraulic damper 400, as described in more detail below with respect to FIG. 4.

In some embodiments, the knee angle circuit board 304 may be encased in an enclosure, coated (e.g., with Parylene C), or overmolded (e.g., in a resin) to protect it from environmental effects and impacts occurring while the prosthetic knee 100 is used or handled. The enclosure, coating or overmold may also advantageously provide electrical isolation of the knee angle circuit board 304.

In some embodiments, the knee angle circuit board 304 may further include a light emitting diode (LED) 310 that may illuminate (e.g., in different patterns or colors) to convey alerts to the use of the prosthetic. The knee angle circuit board 304 may be attached to the side of the lower frame portion 104 of the prosthetic knee 100 at the pivot point 106 between the upper frame portion 102 and lower frame portion 104 of the prosthetic knee 100.

FIG. 4 shows a front view of an example hydraulic damper 400 of the prosthetic knee 100. The hydraulic damper 400 may include a cylinder 405, a piston 410, hydraulic fluid (e.g., glycol ether, organophosphate ester, polyalphaolefin, a propylene glycol, a silicone oil, NaK-7, etc.), and a valve-gear motor system. The piston 410 may move slidably along the length of the cylinder 405 in response to a change in the amount of hydraulic fluid within the cylinder, which is controlled by the valve-gear motor system. Gearmotors 402 and 404 are activated in response to feedback from the knee angle sensor and accordingly rotate the valve cores of valve cartridges 406 and 408, respectively. Motor couplers 407 and 409 connect gearmotors 402 and 404 and valve cartridges 406 and 408, respectively. As the valve cores are rotated by the gearmotors 402, 404 via the motor couplers 407, 409 based on information from the knee angle sensor and other sensors of the prosthetic knee 100 that provide information indicative of the user's gait, the size of a valve orifice in the valve cartridges 406, 408 via which hydraulic fluid passes can be varied, which in turn changes the hydraulic resistance by exerting forces on the piston 410.

In some embodiments, the resistances for the bending and stretching (i.e., flexion and extension) motions of the prosthetic knee 100 may be set separately by individual valve and gearmotor assemblies. For example, when the knee angle is registered as being fully extended (e.g., at 0°-1° of flexion) by the knee angle sensor, the microprocessor will verify with the IMU that the lower frame portion 104 of the prosthetic knee 100 is also tilted forward (e.g., when fully extended and behind the user). This is indicative of a “toe-off” condition. The microprocessor may then command the gearmotor 402 (e.g., the flexion valve) to rotate valve cartridges 406 to a pre-set lower resistance (e.g., swing flexion) allowing the user to achieve heel rise.

The knee angle sensor continuously monitors the knee angle and changes in the knee angle. Once the knee angle sensor detects that the knee angle is no longer increasing (i.e., flexing) and the knee angle is extending again, the microprocessor will command the gearmotor 402 to rotate the valve cartridges 406 (e.g., the flexion valve) back to the pre-set stance flexion resistance to prepare for heel strike. If the knee lower frame portion 104 of the prosthetic knee 100 has been detected as being tilted backwards while the knee angle is fully extended, this is indicative of the heel strike position and the stance flexion resistance settings will remain. If the prosthetic knee 100 is flexed beyond the fully extended threshold of 0°-1°, it will maintain its pre-set stance flexion resistance and will not go into a lower swing flexion resistance even if the prosthetic knee 100 is tilted forwards. Furthermore, to ensure that changes to the valve resistances are only being made when the user is actively walking, the knee angle sensor will monitor for change in the knee angle before changing resistance settings via the valves valve cartridges 406, 408. This advantageously prevents the prosthetic knee 100 from going into a lower resistance state on a false step. Alternate means of correlating the knee angle position to gait patterns may also be used, such as, for example, a look-up table.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A prosthetic joint, comprising:
a frame;
a connector pivotally coupled to a proximal portion of the frame, the connector configured to pivot in an anterior-posterior direction of the frame about a pivot axis that extends in a medial-lateral direction of the frame, the frame and connector being portions of a joint;
a shaft extending along the pivot axis, the shaft extending through the connector and coupled to the frame on opposite sides of the connector;
a sensor attached to a side of the frame at or proximate the pivot axis;
a diametrically polarized magnet housed in a magnet cup, the magnet cup being housed in a hollow portion of the shaft so that the magnet is centered along the pivot axis, the magnet cup separating the magnet from the shaft,
wherein rotation of the connector relative to the frame simultaneously rotates the shaft, the magnet cup and the diametrically polarized magnet, the sensor configured to measure a motion of the connector relative to the shaft by detecting a magnetic field generated by the magnet.

2. The prosthetic joint of claim 1, wherein in the prosthetic joint is a prosthetic knee.

3. The prosthetic joint of claim 2, wherein the prosthetic knee includes a hydraulic damper including a cylinder and piston configured for slidable travel within the cylinder.

4. The prosthetic joint of claim 1, wherein a distal portion of the frame comprises a connector.

5. The prosthetic joint of claim 4, wherein the connector is a pyramid connector.

6. The prosthetic joint of claim 5, wherein the pyramid connector is configured to couple to a prosthetic device.

7. The prosthetic joint of claim 6, wherein the prosthetic device is a socket.

8. The prosthetic joint of claim 1, wherein the sensor is attached to a circuit board.

9. The prosthetic joint of claim 8, wherein the sensor is encased in a coating and/or an overmold and wherein the coating and/or the overmold is configured to electrically isolate the circuit board.

10. The prosthetic joint of claim 9, wherein the overmold is comprised of resin.

11. The prosthetic joint of claim 1, wherein the sensor is encased within an enclosure, an overmold, and/or a coating, the enclosure, the overmold, and/or the coating configured to inhibit exposure of the sensor to environmental conditions.

12. The prosthetic joint of claim 1, wherein the magnet cup is configured to separate the diametrically polarized magnet from the shaft to not inhibit a magnetic field output of the diametrically polarized magnet.

13. The prosthetic joint of claim 1, further comprising a controller, wherein the controller comprises a microprocessor configured to correlate the measured motion to a knee angle.

14. A prosthetic knee comprising:
a hydraulic damper, the hydraulic damper including a cylinder and piston configured for slidable travel within the cylinder;
a frame;
a connector pivotally coupled to a proximal portion of the frame, the connector configured to pivot in an anterior-posterior direction of the frame about a pivot axis that extends in a medial-lateral direction of the frame, the frame and connector being portions of a joint;
a shaft extending along the pivot axis, the shaft extending through the connector and coupled to the frame on opposite sides of the connector;
a sensor attached to a side of the frame at or proximate the pivot axis;
a diametrically polarized magnet housed in a magnet cup, the magnet cup being housed in a hollow portion of the shaft so that the magnet is centered along the pivot axis, the magnet cup separating the magnet from the shaft,
wherein rotation of the connector relative to the frame simultaneously rotates the shaft, the magnet cup and the diametrically polarized magnet, the sensor configured to measure a motion of the connector relative to the shaft by detecting a magnetic field generated by the magnet.

15. The prosthetic knee of claim 14, wherein a distal portion of the frame comprises a connector.

16. The prosthetic knee of claim 15, wherein the connector is a pyramid connector.

17. The prosthetic knee of claim 16, wherein the pyramid connector is configured to couple to a prosthetic device.

18. The prosthetic knee of claim 17, wherein the prosthetic device is a socket.

19. The prosthetic knee of claim 14, wherein the sensor is attached to a circuit board.

20. The prosthetic knee of claim 19, wherein the sensor is encased in a coating and/or an overmold, and wherein the coating and/or the overmold is configured to electrically isolate the circuit board.

21. The prosthetic knee of claim 20, wherein the coating and/or overmold is comprised of resin.

22. The prosthetic knee of claim 14, wherein the sensor is encased within an enclosure, an overmold, and/or a coating, the enclosure, the overmold, and/or the coating configured to inhibit exposure of the sensor to environmental conditions.

23. The prosthetic knee of claim 14, wherein the magnet cup is configured to separate the diametrically polarized magnet from the shaft to not inhibit a magnetic field output of the diametrically polarized magnet.

24. The prosthetic knee of claim 14, further comprising a controller, wherein the controller comprises a microprocessor configured to correlate the measured motion to a knee angle.

25. The prosthetic knee of claim 24, wherein the controller is configured to adjust a hydraulic resistance of the hydraulic damper based on the knee angle corresponding to the motion measured by the sensor.
